(19) United States
(12) Patent Application Publication
(10) Pub. No.: US 2006/0246127 A1
(43) Pub. Date: Nov. 2, 2006

(54) Moulded body made from a gelatine-free material filled with a liquid filling

(76) Inventor: Rüdiger Freier, Flawil (CH)

Correspondence Address: NATH & ASSOCIATES, 112 South West Street, Alexandria, VA, 22314, US

(21) Appl. No.: 10/541,613

(22) PCT Filed: Dec. 29, 2003
(86) PCT No.: PCT/EP03/14950

§ 371 Date: Apr. 14, 2006

(30) Foreign Application Priority Data
Jan. 8, 2003 (EP) 03405005.4

(51) Int. Cl. A61K 9/48; B27N 3/00

(52) U.S. Cl. 424/451; 264/109

(57) ABSTRACT

The invention relates to a moulded body, comprising a shell mass made from gelatine-free material and a liquid as filler, whereby the shell mass comprises at least one first biopolymer and at least one plasticiser. At the time of establishment of equilibrium of the water content between the shell mass and the filler, the filler has a water content of less than 3 wt. %, based on the filler. The invention further relates to a method for production thereof.

The present invention relates to soft capsules which are distinguished by very low water contents in the capsule shell and capsule content after the equilibrium between the two compartments has been established, and are therefore particularly suitable for the administration of water-sensitive or sparingly water-soluble active substances.

The formulation of sparingly water-soluble active substances for administration to humans is a complex and difficult task. Owing to relative water insolubility, often only insufficient bioavailabilities are achieved. By additional micronization of the active substance particles, the kinetics of the formation of a solution can be advantageously influenced. However, the bioavailability may nevertheless be limited. A successful approach is made with the application of nonaqueous solutions of these active substances. The solvents chosen must firstly dissolve the active substance sufficiently and secondly transport the active substance into the aqueous phase or at least to the biomembranes. A basic requirement is that these solvents are also physiologically sufficiently tolerated at the place of application and of release.

Suitable organic solvents are ethanol, tetrahydrofuran (THF), dimethyl sulfoxide (DMSO), propylene glycol, glycerol, polyethylene glycol, ethanol, N-methylpyrrolidone, vitamin E TPGS, compounds of the Tween series and combinations thereof and many others. Tetrahydrofuran and dimethyl sulfoxide are very efficient solvents but are generally unsuitable owing to their toxicity.

The suitable solvents are distinguished by a high solubility product (similis solvuntur), which is generally associated with high to medium polarity, high dielectric constant and low molecular weights (cf. Handbook of Chemistry, CRC Press, 64th edition (1983-1984), page C-696).

The introduction of solutions of active substances with the use of such suitable solvents or mixtures thereof as content into soft capsules whose shell consists of gelatin or other high molecular weight substances leads—owing to the properties of the solvent—to “dissolution” or partial swelling of the polymeric shell material or at least migration of the solvent into the shell on the one hand and, on the other hand, to migration of low molecular weight components from the shell into the capsule content. The equilibrating migration occurs in particular in the case of water but is also to be observed in the case of other components used in the formulation. Examples of such components are glycerol, propylene glycol and similar substances used as plasticizers in the shell or as solvents in the capsule content.

In order to solve this problem, it was proposed, for example in EP-A-0 649 651, to add special hydrophilic substances, such as glycerol, to the shell material. The consequently rapid establishment of equilibrium between the amount of hydrophilic substance in the content and in the shell material should lead to storage-stable molded parts with filling materials. As described in EP-A-0 649651, however, a particular development of the rotary die method is required for the production of such molded parts. According to EP-A-0 649 651, there is therefore a generally not inconsiderable additional technical complexity in the production of molded parts from gelatin for contents with sparingly water-soluble active substances.

It is also known that the migration of water from the shell into a solution which is anhydrous during the encapsulation and comprises polyethylene glycol (PEG) and active substance (e.g. temazepam or nifedipine) causes the active substance, which is more poorly soluble in water than in PEG, to crystallize owing to the reduction of the solubility product, with the result that the bioavailability of the active substance is dramatically reduced. In the case of cyclosporin-containing capsule contents, too, precipitations of active substance by water are known.

This problem occurs frequently in the case of conventionally used gelatin capsules. In the encapsulation of active substance solution in soft gelatin capsules, the shell material has a high water content (25-40%) at the time of encapsulation. The migration of water from the shell into the capsule content is measurable after only a few minutes, depending on the water affinity of the capsule content. Up to the end of the drying of the soft capsule shell, an equilibrium of the water content between shell material and capsule content has been established after a few hours. Nothing changes with regard to this basic state of affairs even when high-bloom and simultaneously low-viscosity gelatin is used (cf. J. P. Stanley in Lachman, Lieberman, Kanig (editors), The theory and practice of industrial Pharmacy, 3^{rd} ed., Lea & Febiger, Philadelphia (1986), page 400).

By adding solvents/plasticizers, such as 1,2-propylene glycol, to the content and at the same time to the shell, it is possible, as described in EP-A-0 649 651, actually to reduce the water content of the gelatin melt but not to eliminate it: EP-A-0 649 651 states that, as a result of the addition of the special hydrophilic components to the shell material, the amount of water for production of the shell material can be reduced. This is the consequence of the constant total plasticizer fraction (organic plasticizer plus water). With an increase in the organic fraction, the proportion of water which is required for an acceptable viscosity of the gelatin melt can in return be reduced. After encapsulation is complete, the water in the shell material must be removed by an additional drying step. This in any case leads to unavoidable migration of water from the shell into the content during the drying period. Serajuddin, Sheen and Augustine (J. Pharmaceutical Sci. 7581) (1986), 62-64) state that the transfer of water from the shell to the content can be reduced by using a soft capsule content which is solid up to 36° C. The solidification of the content after encapsulation leads to a reduction of the diffusion and hence favors the drying process.

EP-A-1 103 254 disclosed starch capsules as an alternative to the conventional gelatin capsules since, with regard to its biodegradability and to the BSE problems associated with gelatin, starch has advantages over gelatin as a raw material and in addition has the physical properties required for the production of soft capsules. Capsules produced therefrom and having a sparingly water-soluble active substance as an ingredient are, however, not described.

It was the object of the present invention to provide an easily preparable dosage form for solutions which contain sparingly water-soluble active substances, no significant precipitation of the active substance occurring even on prolonged storage.

This object is achieved, according to the present invention, by a molded part of gelatin-free material, comprising a shell material of gelatin-free material and a liquid as content, the shell material comprising at least one first biopolymer and at least one plasticizer and, after equilibrium has been established between the water content of the shell and that of the content, the content having a water content of less than 3% by weight, based on the content.

It has surprisingly been found that, with the use of a particular shell material, preferably of starch, and of solutions having a low water content as filling material, it is possible to produce capsules in which no precipitation of a sparingly water-soluble active substance occurs even on prolonged storage. This is presumably due firstly to the low water content of the shell material, and secondly it is caused by the low absorption of water from the environment by the shell material, with the result that there is only very little migration of water from the shell material into the filling material even on prolonged storage.

The very low water content of less than 3% by weight in the molded part content is not achievable with conventional gelatin molded parts. For the production of gelatin molded parts, a certain amount of water is indispensable for dissolving and melting the gelatin. By adding certain hydrophilic substances, such as 1,2-propylene glycol, to the shell material, it is possible, according to EP-A-0 649 651, to reduce the required amount of water, but only at the expense of a complicated production process brought about by the hydrophilic component. Moreover, the water present in the shell material must be removed by an additional drying step. This unavoidably results in a certain migration of the water from the shell material into the molded part content. In the case of active substances which are particularly sparingly water-soluble, this may be unacceptable simply because of the associated precipitation of the active substance.

According to the present invention, it has now been found that, with the use of at least one biopolymer and in particular starch instead of gelatin as the main component of the molded part, the production of shell material and the formation of capsules having a very low water content are possible without a drying step. In the case of the these materials the plastic deformation can be achieved at higher pressures and temperatures than can usually be achieved in capsule production. The danger of migration of water from the shell into the molded part content is thus dramatically reduced. It is thus possible to produce capsules which have a water content of less than 3% by weight, based on the molded part content even after the equilibrium between shell and molded part content has been established. This water content can also be substantially maintained during prolonged storage. Thus, a high stability and bioavailability of the active substance dissolved in the molded part content are brought about.

According to the present invention, establishment of equilibrium is to be understood as meaning the time when the concentration gradient between the concentration of water in the molded part content and the concentration of water in the shell material have equalized so that substantial migration of the water from the shell material into the molded part content, or vice versa, no longer occurs. This time is usually not reached directly after the production of the molded part but only after some time of storage or of drying, if the capsules are subjected to a drying step. The distribution of the water and of the low molecular weight components between shell (pseudosolid phase) and molded part content (liquid phase) is determined not only by the initial concentration but also especially by the solubility, which may be expressed, for example, by the solubility parameter δ (cf. Handbook of Chemistry, CRC Press, 64^{th} edition (1983-1984), page C-696).

Owing to the achievable low water concentration in the shell material according to the invention, there is only very little migration of water into the molded part content from the outset. The amount of water penetrating into the molded part content due to migration is dependent on the water content of the molded part content. The lower the concentration gradient of water between molded part content and shell material at the beginning, the less migration is required for establishing equilibrium. This means on the other hand that the shell material is permitted to contain less water the more water is contained in the molded part content, since otherwise the threshold value of 3% by weight of water in the molded part content after equilibrium has been established may be exceeded due to migration. This applies in an analogous manner to other migrating molecules (i.e. molecules having a molecular weight of less than 300 to 600 g/mol) which occur in the molded part content and/or in the shell material.

As shown below, with a knowledge of the water content of the molded part content, the person skilled in the art can adjust the water content of the shell material as desired by a suitable choice of the process parameters during the production of the molded part, in order to avoid exceeding the threshold value of 3% by weight of water in the molded part content in the molded part after equilibrium has been established. The optimum adjustment of the process parameters can be determined by the person skilled in the art without problems for any shell material according to the invention.

According to the present invention, the shell material comprises at least one biopolymer instead of gelatin. The use of starch as shell material is particularly preferred according to the invention.

According to the present invention the molded part content is a liquid. The term “liquid” is intended here to include solutions, emulsions and dispersions.

The term starch is to be understood as meaning natural starches and physically and/or chemically modified starches. According to the present invention, the starches which are described in EP-A-1 103 254 and to whose disclosure in this context reference is hereby expressly made can be used as shell material. This is preferably starch whose amylopectin content is more than 50%, based on the total weight of the anhydrous starch. Physically and/or chemically modified potato starch have proven to be particularly preferable.

However, in the widest sense, all polyglucans, i.e. 1,4- and/or 1,6-poly-α-D-glucans, and/or mixtures of these are suitable for the present invention.

In a preferred embodiment, the starch is a hydroxypropylated starch. The degree of substitution (DS) is in the range from 0.01 to 0.5, preferably in the range from 0.05 to 0.25 and more preferably in the range from 0.1 to 0.15. In particular, it is a hydroxypropylated potato starch.

In a further preferred embodiment, the starch is pregelatinized starch. Above a temperature which is typical for every type of starch, “solution” of the starch particles, i.e. irreversible disintegration of the starch particles, occurs in aqueous starch suspensions after a maximum degree of swelling has been reached. This process is also referred to as “gelatinization”. The gelatinization, i.e. the irreversible swelling of the starch particles at relatively high temperature up to 40 times the original volume is based on gradual water absorption and breaking the hydrogen bridge bonds, which permits further hydration up to complete disintegration of the starch particle structure. The gelatinization can also be effected at low water concentrations at relatively high pressures and temperatures and with simultaneous use of plasticizers, such as, for example, glycerol.

The use of a pregelatinized starch whose amylopectin content is more than 50%, based on the total weight of the anhydrous starch and which, after processing according to the invention to give a homogenized material, has a Staudinger index of at least 40 ml/g, preferably at least 50 ml/g and more preferably at least 80 ml/g is most preferred according to the invention.

According to the present invention, the shell material may also contain other physically and/or chemically modified biopolymers apart from starch, in particular polysaccharides and plant polypeptides. The group consisting of the physically and/or chemically modified biopolymers includes, inter alia, cellulose ethers and cellulose esters (e.g. hydroxypropylcellulose, cellulose acetate), alginates (alginic acid and salts), carrageenans (lambda, iota, kappa), agar, pectins and pectin derivatives from various fruits (e.g. apple, lemons, etc), galactomannans (e.g. guar and carob bean meal), glucomannans (e.g. konjac), arabinogalactans, plant gums (acacia gum, tragacanth, karaya, tamarind), gums from microorganisms, such as bacteria and fungi (e.g. xanthan), amino sugar derivatives (such chitosan, chitin), proteins (casein, zein, gluten, etc), dextrins, maltodextrins, cyclodextrins, gellan, pullulan and synthetic or “debranched” starch. Furthermore, the shell material may also comprise physiologically tolerated synthetic polymers, such as, for example, PVP (polyvinylpyrrolidone), PVA (polyvinyl alcohol), polyvinyl acetate, PLA (polyacetic acid) or acrylic esters.

The mixture used for the production of the molded part according to the invention preferably contains the biopolymer, preferably starch, in a weight range from 45 to 80% by weight, based on the total weight of the mixture. The biopolymers used, in particular the starch described above, should, according to the invention, preferably have a moisture content of from 1 to 30%, preferably from 15 to 23%. This facilitates the establishment of the water content of the molded part content by the method described below.

The shell material according to the invention must comprise at least one plasticizer. Preferably used plasticizers are those which have a solubility parameter equal to or >16.3 (MPa)^{1/2}. The organic plasticizers are selected from the group consisting of polyalcohols, organic acids, amines, acid amides and sulfoxides. Polyalcohols are preferred. Examples of plasticizers which may be used according to the invention are glycerol, syrup of hydrogenated, polyalcohol-containing starch degradation products, starch degradation products (containing oligosaccharides, di- and monosaccharides), sorbitol, maltitol, mannitol, erythritol, xylitol, propylene glycol, polyglycerols, polysorbitans, polyethylene glycols, polyethylene/polypropylene polymers, sorbitan fatty acid esters, N-methylpyrrolidone and mixtures thereof. By definition, a syrup must contain at least 70% w/w dextrose equivalents (regulation on sugar types of the Federal Republic of Germany of Apr. 24, 1993 (BGBl. [Federal German Law Gazette] I, page 512). The syrup used in the present invention contains from 10 to 30%, preferably from 15 to 30%, of water. In a particular embodiment, an excess of at least 1 percent by weight, relative to other plasticizers, of a polyol originating from hydrolyzed and hydrogenated starch is present. According to the present invention, however, the water content of the mixture used as starting material should be in the range from 6 to 30% by weight, based on the total mixture. The total plasticizer content of the mixture used for the preparation of the shell material according to the invention is at least 12% by weight, based on the weight of the anhydrous starch. In a preferred embodiment, the content of the plasticizer is in a range from 30% by weight to 60% by weight and more preferably in a range from 38% by weight to 55% by weight, based on the total weight of the material.

The total content of plasticizer (i.e. the content of water (in starch and plasticizer formulation) and of plasticizer) is equally of importance for the sorption, strength, the modulus of elasticity and the melt flow rate at elevated and normal temperatures. It is preferable that not only polyols of a pure, chemical nature (such as glycerol and sorbitol) are used, but particularly polyol mixtures (including higher molecular weight forms), all of which are prepared from enzymatic and/or chemical hydrolysis of starch, followed by hydrogenation. In most cases, these products cannot be sold as dry substances since, owing to the different chemical properties and the chain length of the molecules contained, no crystallization can take place. The water content of the syrup to be used (preferably a mixture of sorbitol/mannitol syrup) must be taken into account for the entire method according to the invention.

Depending on the required properties of the resulting molded part, at least one additive in a weight range from 3.5% by weight to 15% by weight, preferably from 5% by weight to 8% by weight, based on the total weight of the mixture may also be added to the mixture used for the preparation of the shell material according to the invention. The additives are selected from the group consisting of carbonates and bicarbonates of the alkali metal and alkaline earth metal ions, further disintegration assistants, molded part contents, dyes and antioxidants.

The homogenized material is preferably rendered opaque by adding titanium dioxide as a molded part content.

Calcium carbonate and amylases are preferably added as a disintegration assistant for rapid disintegration of the capsule shell.

In a preferred embodiment, the mixture used for the preparation of the shell material according to the invention additionally contains an internal lubricant and mold release agent, which is selected from the group consisting of lecithins, mono-, di- or triglycerides of edible fatty acids, polyglyceryl esters of edible fatty acids, polyethylene glycol esters of edible fatty acids, sugar esters of edible fatty acids, and edible fatty acids and the alkali metal and alkaline earth metal salts thereof and combinations thereof. The lubricant and mold release agent is preferably contained in the mixture in a range from 0 to 4% by weight, based on the total weight of the mixture. It is preferably added to the mixture in an amount of from 0.5 to 2% by weight and more preferably from 0.8 to 1.5% by weight. The lubricant and mold release agent is advantageously selected from the group consisting of glyceryl monostearate and lecithin.

Edible fatty acids are understood as meaning the monocarboxylic acids occurring as acid components of the triglycerides of natural fats. They have an even number of carbon atoms and have a straight-chain carbon skeleton. The chain length of the fatty acids varies from 2 to 26 carbon atoms. A group of these fatty acids which is preferred according to the invention comprises saturated fatty acids.

In order further to reduce the in any case low absorption of water from the environment by the shell material according to the invention, the molded parts according to the invention may additionally be coated. This coating can, however, also be effected for other purposes, for example to retard the release of active substances, to provide resistance to gastric fluid, to provide aroma protection, for esthetic purposes, such as for providing gloss or color, but also for keeping the moisture content of the capsule material constant so that these do not become brittle or tacky and retain their physical properties. Such coatings may consist of waxes, resins, gums and/or lipids and/or synthetic polymers having a hydrophobic character and are selected from the group consisting of beeswax (E901), Carnauba wax (E903), candelilla wax (E902), berry wax, montan glycol wax (E912), polyethylene glycol wax oxidation products (E914), montanic acid esters (E912), rosin esters, shellac (E904), mono-, di- and triglycerides of edible fatty acids (E471, sugar esters of edible fatty acids (E476), dimethylpolysiloxane (E900), acrylic esters (e.g. Eudragit), cellulose ethers (e.g. ethylcellulose (EC)) and cellulose esters (e.g. HPMC) and derivatives thereof.

The molded part according to the invention contains a contains a single-phase to multi-phase molded part content (emulsion, preemulsion, suspension, solution) having a liquid to pasty consistency. The molded parts according to the invention are suitable in particular for molded part contents which comprise at least one active substance which is sparingly water-soluble but is homogeneously dissolved or emulsified in the molded part content. According to the present invention, a sparingly water-soluble active substance is to be understood as meaning a pharmacologically active substance or a substance having a cosmetic effect or a substance used as a food supplement, which has a solubility in water of less than 1% (w/v). The following may be mentioned as examples of such sparingly water-soluble active substances: the class consisting of cyclosporins, such as, for example, cyclosporin A, macrolides, such as, for example, rapamycin, paclitaxel, certain vitamins, flavonoids, coenzyme Q10 or isotretinoin, ibuprofen, temazepam, nifedipine, nimodipine, paracetamol or codeine. Furthermore, the molded parts according to the invention are suitable for molded part contents which contain water-sensitive active substances.

The filled bodies according to the invention may contain one or more such active substances.

In the molded parts according to the invention, these active substances may be present in the formulations which are usually used for sparingly water-soluble compounds. The formulations mentioned at the outset, in customary organic solvents, such as ethanol or polyethylene glycol, may be mentioned as examples for this purpose. However, it is also possible to use complex vehicles as solvents for such active substances. The microemulsions or premicroemulsion concentrates mentioned in EP-A-0 649 651 may be mentioned as an example. The microemulsions or premicroemulsion concentrates described in WO 01/28518 and WO 01/28520, in particular with cyclosporins or coenzyme Q10 as active substance, are preferred according to the invention. Reference is hereby expressly made to the corresponding content of these documents.

According to the invention, the vehicle for dissolving the sparingly water-soluble active substance is preferably a hydrophilic matrix. Examples are matrices which comprise one or more substances from the group consisting of ethanol, polyethylene glycol, such as PEG 400, or glycerol. However, small amounts of water may also be contained. However, it is of course self-evident that this vehicle must have a water content such that, after equilibrium has been established, the water content in the total molded part content is less than 3% by weight. Vehicles or molded part contents which have a water content equal to or less than 2% by weight, based on the total molded part content, at the time of capsule filling are therefore preferred according to the invention.

The present invention furthermore relates to a method for the production of a molded part described above, comprising the steps:
a) mixing of at least one first biopolymer in the form of powder or granules with at least one plasticizer in liquid form, in particular in the form of a syrup, optionally together with additives, to give a homogeneous raw material mixture;
b) melting of the raw material mixture with supply of heat and under superatmospheric pressure in a processing apparatus, in particular in an extrusion stage, to give a thermoplastically processible material;
c) optionally preparation of an intermediate, in particular of granules, after cooling of the material obtained in step b), and further treatment to give a thermoplastically processible material;
d) formation of at least one film of the thermoplastically processible material according to step b) or optionally step c), in particular by extrusion from a slot die;
e) production of molded parts with the use of the film in an intermittent or continuous method at a molded part station, in particular on a rotary die encapsulation machine, and with filling of the molded part with a liquid as molded part content;
   it not being necessary to subject the complete molded part to a drying process after leaving the molded part station.

This method is described in detail in EP-A-1 103 254. References here are expressly made to the content thereof in this context.

The terms “thermoplastically processible”, “melt”, “syrup” and “amorphous” are used in the present application according to the definition in Rompp Chemie Lexikon [Römpp Chemistry Lexicon], editors: J. Falbe, M. Regitz, 9th edition, 1992, Georg Thieme Verlag, Stuttgart.

The conversion of a starch-containing mixture into the thermoplastic, preferably homogenized state in step b), as well as the subsequent processing steps, must be effected under conditions which prevent uncontrolled degradation of the amylose and amylopectin molecules to short fragments. The cooperation of all processing parameters, such as, for example, temperature, pressure, residence time and kneading power, must be taken into account during the various steps in order to prevent substantial degradation of the starch molecules. Thus, substantial degradation of the starch molecules can be avoided, for example even at relatively high temperatures, if the residence times of the starch-containing material at these temperatures is kept short.

In a preferred embodiment, the temperature of the material in the first and optionally second processing apparatus, and during production of the material extrudate, does not exceed 160° C., preferably 140° C., more preferably 120° C. and most advantageously 90° C. At 160° C., the digestion process in step a) should in particular be complete in less than 5 minutes, preferably less than 3 minutes.

In a further preferred embodiment, the energy introduced into the material by kneading for producing a thermoplastically processible homogenized material in step a) to c) does not exceed 0.3 kWh/kg, preferably 0.2 kWh/kg and more preferably 0.175 kWh/kg.

The formation of the film in step d) is preferably effected by extrusion under a pressure of more than 5·10⁴ Pa and at a temperature of from 80° to 105° Celsius from a slot die in to an atmospheric environment.

The conversion into the thermoplastically processible state results in irreversible swelling of the starch particles, which is required in order for the material to be capable of being converted into the homogeneous state or to be present in the homogenized state after cooling. Furthermore, by means of steps a) to c), a material which also substantially no longer has any natural ordered regions in the starch is produced. Natural ordered regions lead to gel formation in the material extrudate, i.e. to inhomogeneities which have a particularly disadvantageous effect when the material extrudate in step c) is an extruded film. “Substantially no natural ordered regions” is intended to mean that these have been destroyed to such an extent that impairment of the physical parameters of the extruded material which are relevant with regard to the forming cannot be attributed to the presence of natural ordered regions.

The term “homogeneous material” or “homogenized material” is therefore to be understood as meaning a material which has substantially identical physical properties (parameters) at every point in the material. Slight differences may occur at the respective material or molded part surfaces as a result of absorption of atmospheric humidity. The material is present in homogeneous or homogenized form when, under the microscope, the number of still visible starch particles is on average less than one percent. For this purpose, the material is cooled in the thermoplastic state, cut into thin disks and analyzed under an optical microscope.

A homogenized material is obtained by converting a corresponding starting mixture into a softened or even liquid, thermoplastically processible state. The major part of the components accounting for the mixture (starch, organic plasticizer, lubricant and mold release agent) may be present in the molten state and, with a sufficiently long standing and/or mixing (kneading) time, the material will have substantially the same properties or chemical composition at every point of the melt (homogeneous material). This homogeneous state is also retained during and after cooling of the thermoplastic state. No separation processes occur. This ensures uniform mechanical properties of the molded part at room temperature.

As mentioned above, the water content of the mixture used in step a) can be modified in step b) or c) in the method according to the invention in a specific manner well known to the person skilled in the art. The physical parameters, which are dependent on the water content may thus be subjected to changes. An additional drying step after production of the molded parts according to the invention is complete is therefore no longer necessary. The modification of the water content of the mixture in step b) or c) can be carried out according to the invention preferably by controlled release of water vapor in a decompression zone from the processing apparatus or by introducing water in an injection zone into the processing apparatus. In the case of the twin-screw extrusion preferred according to the invention, one or more so-called devolatilization zones are used, via which the excess water (from the biopolymer, such as, for example, the starch, and/or the plasticizer, for example the polyol syrup) is removed from the melt. By adjusting the reduced pressure effective in the melt (for example with the aid of the fresh air valve between vacuum pump and extruder), the final water content of the extruded material can be established specifically. With a fixed total throughput, for example at an absolute pressure of 200 mbar effective in the devolatilization zones, it is ensured that the final water content of the extrudate is, for example, 6%; if the pressure is adjusted to 450 mbar, the final water content of, for example, 12% results.

According to a preferred embodiment of the present invention, the film formed in step d) has a Staudinger index of at least 40 ml/g, preferably at least 50 ml/g and more preferably at least 80 ml/g. Regarding the explanations of the term Staudinger index and the determination thereof, reference is made to the entire content of EP-A-1 103 254 in this context, which is hereby expressly incorporated by reference.

The materials according to the invention which are obtained by the preparation method according to the invention have mechanical properties, such as, for example, ε_{B}, σₘ, E, which are less dependent on the temperature in the temperature range from about 20° C. to about 80° C. than outside this range. The so-called rubber plateau is of decisive importance for the forming and filling of the films into filled molded parts. Thus, Young's modulus of elasticity E of the starch-containing film according to the invention is not more than 2 MPa, preferably not more than 1 MPa, at the moment of forming and filling in the rotary die process. In other words, the film must not oppose the filling pressure of the filling material, which in the end is responsible for forming the capsule shell in the rotary die process, at the filling wedge contact pressure generated by the machine, with a resistance such that the filling material runs out between film and filling wedge. The processibility of the films produced from these materials to soft capsules in the rotary die method is permitted thereby.

By means of the procedure according to the invention it is possible substantially to exclude highly degraded oligomers of starch. This makes it possible to incorporate large total amounts of plasticizers into the material.

The extruded bands are either directly further processed or optionally wound on rolls for storage, for example, with the use of plastic films as an intermediate layer. Polyethylene has proven to be the most suitable film material.

The process for forming the material extrudate to give a molded part, in particular the forming of an extruded film into a one-part soft capsule by the methods known in industry, requires elongations at break of the material extrudate, in particular of the film, of at least 100% in the range from 40° C. to 90° C., preferably from 60° C. to 80° C. In a preferred embodiment, the elongation at break of the material extrudate, in particular of the film, is at least 160% and more preferably at least 240%.

The strength σₘ of the material extrudate, in particular of the molded part produced therefrom, should be at least 2 MPa at 25° C. and 60% relative humidity. In a preferred embodiment, σₘ is greater than or equal to 3.5 MPa and more preferably greater than or equal to 5 MPa. At room temperature, this value ensures sufficient stability of the capsule shell (packaging, storage, transport safety and use).

However, the filling is effected at elevated temperatures of the film, which necessitates a filling pressure of not more than 2 MPa. This is the case for the present material having a Young's modulus of elasticity E of less than or equal to 2 MPa at the preferred encapsulation temperature (40° C. to 90° C.).

The film obtained by means of the method according to the invention, in step d), can be processed in particular for the production of soft capsules on all units known in industry and intended for the production of one-part capsules. Continuous units and in particular the rotary die process have proven to be particularly suitable. The capsule wall is welded under the action of heat, preferably at a temperature greater than or equal to 50° C., from two molded part halves punched beforehand from a film. Two “continuous films” are passed through two adjacent, counter-rotating rollers or rolls having recesses. While the starch film is pressed by the filling pressure of the molded part content into the recess and the capsule halves are thus formed, the pumpable and sprayable capsule filling is exactly metered by means of a valve and introduced by means of a filling wedge into the draw-in-angle of the shaping rolls. The shape and size of the capsule is thus dependent on the geometrical dimensions of the recesses in the rolls and the full volume metered in.

The molded part formed in step e), preferably with the aid of the rotary die process, can be filled with the liquid, pasty or molten molded part content described above.

Consequently, the term capsule is therefore to be understood as meaning not only the typical capsule shapes but also any other possible shape of “shells”, such as, for example, spheres, cushions and figures. There are to date numerous further developments of and deviations from this basic principle.

The coextrusion, coating and the lamination of the molded part according to the invention with materials whose film-forming property is based on synthetic and/or natural polymers creates additional possibilities for providing certain properties of the capsule shell by means of a multilayer film,

In particular, by means of the multilayer structure, it is possible to produce a molded part which has a readily weldable coating on the inside while the outside is coated in such a way that there is a retardant effect on the disintegration or gastric fluid resistance of the capsules.

The shell materials according to the invention are suitable for the production of multichamber or two-chamber capsules, as described, for example, in WO 00/28976. Since the water content of the film or of the films can be adjusted to be low, virtually no stresses occur in the finished dried capsules, in particular in the partitions forming the chambers, which substantially increases the stability of the multichamber capsules in comparison with soft gelatin multichamber capsules.

The molded part, in particular the capsule shell, has a thickness in the range from 0.1 to 2 mm, preferably from 0.2 to 0.6 mm.

The present invention is explained in more detail below with reference to nonlimiting examples. The molded parts were produced by the method and using the apparatus which are explained in detail in EP-A-1 103 254 in FIGS. 3 and 4 and the corresponding passages in the description (sections [0087] to [0093].

The disclosure of EP-A-1 103 254 in this context is hereby expressly incorporated by reference. A twin-screw extruder of the type ZSK30 from Werner & Pfleiderer or of the type ZSK 25 from Krupp, Werner & Pfleiderer was used. All data below are expressed in parts by weight. In all examples the starch described in example 1 was used.

### EXAMPLE 1

The following components were metered continuously via a twin-screw extruder (type ZSK 25, Krupp, Werner & Pfleiderer) and converted into the thermoplastically processible state:

Composition at the beginning of the method:

| | |
|---|---|
| Hydroxypropylated potato starch (natural granular | 60.2 parts |
| form, 21% moisture (absolute water content), 0.1 mol | |
| % of hydroxypropyl groups) | |
| Sorbitol syrup FCCIII (70% solids content) | 37.5 parts |
| Liquid lecithin FCCIII | 1.1 parts |
| Glyceryl monostearate (E471) | 1.2 parts |
| Total amount of water at beginning of extrusion: | 23.8% |
| Water content of the composition at the end of the | 11.5% |
| method in equilibrium with the ambient atmospheric | |
| humidity (25%° C., 60% RH; water content measured | |
| by Karl Fischer method): | |

### EXAMPLE 2 AND COMPARATIVE EXAMPLES 1 AND 2

For the preparation of the molded part content, polyethylene glycol (PEG) 400 (Macrogol 400) and optionally propylene glycol were mixed in each case homogeneously at 20° C. The active substance temapezam was then stirred in at 20-30° C. until completely dissolved.

For comparative examples 1 and 2, the gelatin was introduced into a mixture of water and glycerol at 70° C. and stirred until a viscous, homogeneous melt formed. This was formed into a 0.7 mm thick band on a chill roll at 60° C. The encapsulation was effected using the rotary die technique. The shaped soft gelatin capsules were dried to constant weight at 20-30° C. in dry air having a moisture content of less than 30% (about 18 h).

In example 2, the shell material stated below was melted at 95-105° C. in a single-screw extruder and formed into a 0.7 mm thick band. The encapsulation was effected using the rotary die technique. The shaped soft capsules were conditioned for about 18 h at 20-30° C. in air having a moisture content of about 50%.

Table 1 below shows the different compositions (based on the dry substance):

TABLE 1
Compositions of the molded part content and of the soft capsules

| | Comparative | | Comparative | | | |
|---|---|---|---|---|---|---|
| Component | example 1 | | example 2 | | Example 2 | |
| Filler | | | | | | |
| Temapezam | 20 | mg | 20 | mg | 20 | mg |
| PEG 400 | 470 | mg | 470 | mg | 470 | mg |
| 1,2-Propylene glycol | — | | 43 | mg | 43 | mg |
| Fill weight | 490 | mg | 533 | mg | 533 | mg |
| Shell material | | | | | | |
| Gelatin (dry substance) | 120 | mg | 120 | mg | — | |
| Glycerol (98-101%) | 76 | mg | 76 | mg | 16 | mg |
| Water | 102 | mg | 102 | mg | 28 | mg |
| Modified starch (dry substance) | — | | — | | 160 | mg |
| Sorbitol syrup (dry substance) | — | | — | | 47 | mg |
| Maltitol syrup (dry substance) | — | | — | | 31 | mg |
| Glyceryl monostearate | — | | — | | 3 | mg |
| Shell weight on encapsulation | 298 | | 298 | | 285 | |
| Shell weight after drying/ | 200 | | 200 | | 285 | |
| conditioning | | | | | | |

The water content of the molded part content was determined as described above after 24 hours. The results are shown in table 2. It is evident that a substantially lower final concentration of water in the molded part content is obtained in example 2. The addition of the polar 1,2-propylene glycol had virtually no effect on the result.

TABLE 2
Water content of the molded part content after 24 h

| | Comparative | Comparative | |
|---|---|---|---|
| | example 1 | example 2 | Example 2 |
| Water content in the molded | 5.58% | 5.88% | 2.05% |
| part content after 24 h | | | |

### EXAMPLE 3 AND COMPARATIVE EXAMPLE 3

Cyclosporin was dissolved at 25° C. in ethanol and stirred into a molten mixture of the remaining molded part content components stated in table 3, which mixture had been cooled to 25° C.

For comparative example 3, the gelatin was introduced into a mixture of water and glycerol and sorbitol syrup at 70° C. and stirred until a viscous, homogeneous melt formed. This was formed into a 0.7 mm thick band at 60° C. on a chill roll. The encapsulation was effected using a rotary die technique. The shaped soft gelatin capsules were dried to constant weight at 20-30° C. in dry air having a moisture content of less than 30% (about 18 h).

In example 3, the shell material stated below was melted at 95-105° C. in a single-screw extruder and formed into a 0.7 mm thick band. The encapsulation was effected using the rotary die technique. The shaped soft capsules were conditioned at 20-30° C. in air having a moisture content of about 50% for about 18 h.

Table 3 below shows the different compositions (based on the dry substance):

TABLE 3
Compositions of the molded part content and of the soft capsules

| | Comparative | | | |
|---|---|---|---|---|
| | example 3 | | Example 3 | |
| Filler | | | | |
| Cyclosporin | 25 | mg | 64 | mg |
| Vitamin E polyethylene glycol succinate | 76 | mg | 195 | mg |
| PEG (polyethylene glycol) 400 | 50 | mg | 128 | mg |
| Cremophor RH 40 | 50 | mg | 128 | mg |
| Ethanol absolute | 55 | mg | 141 | mg |
| Fill weight | 256 | mg | 656 | mg |
| Shell material | | | | |
| Gelatin (dry substance) | 113 | mg | — | |
| Sorbitol syrup (dry substance) | 28 | mg | 43 | mg |
| Glycerol (98-101%) | 4 | mg | 17 | mg |
| Water (purified) | 79 | mg | 28 | mg |
| Starch (dry substance) | — | | 148 | mg |
| Maltitol syrup (dry substance) | — | | 29 | mg |
| Glyceryl monostearate | — | | 3 | mg |
| 1,2-Propylene glycol | — | | 7 | mg |
| Shell weight on encapsulation | 224 | mg | 275 | mg |
| Shell weight after drying/conditioning | 140 | mg | 275 | mg |

The water content of the molded part content was determined as described above after 24 hours. The results are shown in table 4. In contrast to comparative example 3, there is a reduction in the water concentration in example 3 here. This is particularly remarkable because a larger amount of very polar, hygroscopic molded part content was encapsulated with a shell having a similar weight at the time of encapsulation in example 3.

TABLE 4
Water content of the molded part content after 24 h

| | Comparative example 3 | Example 3 |
|---|---|---|
| Water content in the molded part | 6.6% | 2.2% |
| content after 24 h | | |

### EXAMPLE 4 AND COMPARATIVE EXAMPLE 4

A molded part content which is not very hydrophilic and comprises poloxamer (polyoxyethylene-12-polyoxypropylene-20-block polymer, MG=2200) 124, Cremophor RH 40 and propylene glycol was heated to about 40° C. 50% of the active substance ibuprofen were introduced and dissolved. The mixture was then cooled to 25° C. with stirring, and the remaining 59% of ibuprofen were added. A finely crystalline suspension was obtained.

For comparative example 4, the gelatin was introduced into a mixture of water and glycerol at 70° C. and stirred until a viscous, homogeneous melt formed. This was formed into a 0.7 mm thick band at 60° C. on a chill roll. The encapsulation was effected using the rotary die technique. The shaped soft gelatin capsules were dried to constant weight at 20-30° C. in dry air having a moisture content of less than 30% (about 18 h).

In example 4, the shell material stated below was melted at 95-105° C. in a single-screw extruder and formed into a 0.7 mm thick band. The encapsulation was effected using the rotary die technique. The shaped soft capsules were conditioned at 20-30° C. in air having a moisture content of about 50% for about 18 h.

Table 5 below shows the different compositions (based on the dry substance). The stated amounts are in percent by weight:

TABLE 5
Compositions of the molded part content and of the soft capsules

| | Comparative | | | |
|---|---|---|---|---|
| | example 4 | | Example 6 | |
| Filler (total weight 792 mg) | | | | |
| Ibuprofen | 400 | mg | 400 | mg |
| Poloxamer 124 | 347 | mg | 347 | mg |
| Propylene glycol | 8 | mg | 8 | mg |
| Cremophor RH 40 | 37 | mg | 37 | mg |
| Shell material | | | | |
| Gelatin (dry substance) | 186 | mg | — | |
| Sorbitol syrup (dry substance) | — | | 53 | mg |
| Glycerol (98-101%) | 110 | mg | 21 | mg |
| Water | 174 | mg | 34 | mg |
| Starch (dry substance) | — | | 183 | mg |
| Maltitol syrup (dry substance) | — | | 36 | mg |
| Glyceryl monostearate | — | | 4 | mg |
| Propylene glycol | — | | 9 | mg |
| Shell weight on encapsulation | 470 | mg | 340 | mg |
| Shell weight after drying/conditioning | 340 | mg | 340 | mg |

The water content of the molded part content was determined as described above after 24 hours. The results are shown in table 6. In this comparison, it is found that an effect is still evident even in the case of a molded part content which is not very hygroscopic.

TABLE 6
Water content of the molded part content after 24 h

| | Comparative example 4 | Example 6 |
|---|---|---|
| Water content in the molded part | 1.8% | 1.6% |
| content after 24 h | | |

1. A molded part comprising a shell material of gelatin-free material and a liquid as molded part content, the shell material comprising at least one first biopolymer and at least one plasticizer, and the molded part content having a water content of less than 3% by weight, based on the molded part content, at the time when equilibrium is established between the water content of the shell material and that of the molded part content.

2. The molded part as claimed in claim 1, wherein the first biopolymer is starch.

3. The molded part as claimed in claim 2, wherein the starch has an amylopectin content of at least 50% by weight, based on the weight of the anhydrous starch.

4. The molded part as claimed in claim 1, wherein the plasticizer is selected from the group consisting of glycerol, syrup of hydrogenated, polyalcohol-containing starch degradation products, sorbitol, maltitol, mannitol, erythritol, xylitol, traces of reducing sugar, propylene glycol, polyglycerol, polysorbitans, polyethylene glycol, polyethylene/polypropylene polymers, sorbitan fatty acid esters, N-methylpyrrolidone and mixtures thereof.

5. The molded part as claimed in claim 4, wherein the plasticizer is a polyol syrup having a water content of from 15% to 30%.

6. The molded part as claimed in claim 1, wherein the shell material furthermore contains at least one further biopolymer which is selected from the group consisting of starch, modified starch, cellulose, in particular partly hydroxypropylated cellulose, alginates, pectins, agar, carrageenan (lambda-, iota- or kappa-carrageenan), galactomannans (guar and carob bean meal), xanthan gum, tamarind, tragacanth gum, karaya gum, chitosan, glucomannans, casein, dextrins, maltodextrins, cyclodextrins, pullulan and arabinogalactan.

7. The molded part as claimed in claim 1, wherein the shell material furthermore contains additives.

8. The molded part as claimed in claim 1, wherein the surface of the molded part is coated with a lipophilic, waxy or polymeric sealing substance.

9. The molded part as claimed in claim 8, wherein the sealing substance is selected from the group consisting of beeswax (E901), carnauba wax (E903), candellila wax (E902), berry wax, montan glycol wax (E912), polyethylene glycol wax oxidation products (E914), montanic acid esters (E912), shellac (E904), mono-, di- and triglycerides of edible fatty acids (E471, sugar esters of edible fatty acids (E476), dimetylpolysiloxane (E900), acrylic esters, cellulose esters and cellulose ethers and derivatives thereof.

10. The molded part as claimed in claim 1, wherein the molded part content comprises at least one sparingly water-soluble active substance.

11. The molded part as claimed in claim 1, wherein the molded part content comprises at least one watersensitive active substance.

12. The molded part as claimed in claim 10, wherein the active substance or substances is selected from the group consisting of cyclosporin, isotretinoin, ibuprofen, temazepam, nifedipine, nimodipine, paracetamol or codeine.

13. The molded part as claimed in claim 1, wherein the active substance or substances is dissolved in a hydrophilic matrix which has a water content equal to or less than 2% by weight, based on the total molded part content.

14. The molded part as claimed in claim 1, wherein the molded part is a soft capsule.

15. The molded part as claimed in claim 1, wherein the molded part is a multichamber capsule, preferably a two-chamber capsule.

16. A method for the production of a molded part as claimed in claim 1, comprising the steps:
a) mixing of at least one first biopolymer in the form of powder or granules with at least one plasticizer in liquid form, in particular in the form of a syrup, optionally together with additives, to give a homogeneous raw material mixture;
b) melting of the raw material mixture with supply of heat and under superatmospheric pressure in a processing apparatus, in particular in an extrusion stage, to give a thermoplastically processible material;
c) optionally preparation of an intermediate, in particular of granules, after cooling of the material obtained in step b), and further treatment to give a thermoplastically processible material;
d) formation of at least one film of the thermoplastically processible material according to step b) or optionally step c), in particular by extrusion from a slot die;
e) production of molded parts with the use of the film in an intermittent or continuous method at a molded part station, in particular on a rotary die encapsulation machine, and with filling of the molded part with a liquid as molded part content;
wherein the completed molded part is not subjected to a drying process after leaving the molded part station.

17. The method as claimed in claim 16, wherein the melting of the raw material mixture in step b) is preferably effected at a temperature of from 80° to 160° Celsius and wherein the pressure corresponds at least to the vapor pressure at this temperature, steam being discharged from the processing apparatus in a decompression zone or water being injected into the processing apparatus in an injection zone.

18. The method as claimed in claim 16, wherein the formation of the film in step d) is effected by extrusion under a pressure of more than 5·10⁴ Pa and at a temperature of from 80° to 105° Celsius from a slot die into an atmospheric environment.

19. The method as claimed in claim 16, wherein a film having a layer thickness of from 0.2 mm to 2 mm is formed in step d).

20. The method as claimed in claim 16, wherein the film formed in step d) has a Staudinger index of at least 40 ml/g, preferably at least 50 ml/g and more preferably at least 80 ml/g.

21. The method as claimed in claim 16, wherein the first biopolymer used in step a) is starch.

22. The method as claimed in claim 21, wherein the starch is a starch or modified starch in natural or crystalline form which has not been destructured.

23. The method as claimed in claim 21, wherein the starch has an amylopectin content of at least 50% by weight, based on the weight of the anhydrous starch.

24. The method as claimed in claim 21, wherein the starch has a moisture content of from 10% by weight to 30% by weight, preferably from 15% by weight to 23% by weight.

25. A molded part obtainable by the method as claimed in claim 16.

26. The molded part as claimed in claim 25, wherein the molded part is a soft capsule.

27. The molded part as claimed in claim 25, wherein the molded part is a multichamber capsule, preferably a two-chamber capsule.

28. The use of a molded part as claimed in claim 1 for the production of storage-stable medicinal products, containing a sparingly water-soluble or water-sensitive active substance.
